(19) United States
(12) Patent Application Publication
(10) Pub. No.: US 2022/0371938 A1
(43) Pub. Date: Nov. 24, 2022

(54) GLASS HAVING HIGH UV TRANSMITTANCE AND HIGH SOLARIZATION RESISTANCE

(71) Applicant: SCHOTT AG, Mainz (DE)

(72) Inventors: Malte Grimm, Mitterteich (DE); Rainer Eichholz, Frankfurt am Main (DE); Josef Rasp, Waldsassen (DE)

(73) Assignee: SCHOTT AG, Mainz (DE)

(21) Appl. No.: 17/748,438

(22) Filed: May 19, 2022

(30) Foreign Application Priority Data
May 21, 2021 (EP) 21175364.5

(51) Int. Cl. C03C 3/083

(52) U.S. Cl. CPC C03C 3/083; C03C 2204/00

(57) ABSTRACT

A glass includes SiO₂ in an amount of at least 60.0 wt.-% and an amount of MoO₃ from 0.1 ppm to 30.0 ppm. Molybdenum is present in such oxidation states that a transmittance at a wavelength of 260 nm is at least 65.0% at a reference thickness of 1.0 mm.

### CROSS REFERENCE TO RELATED APPLICATIONS

This application claims priority to European Patent Application No. 21175364.5 filed on May 21, 2021, which is incorporated in its entirety herein by reference.

### BACKGROUND OF THE INVENTION

### 1. Field of the Invention

The present invention relates to a glass having high UV transmittance and high solarization resistance.

### 2. Description of the Related Art

Short-wavelength ultraviolet (UVC) light is used to kill or inactivate microorganisms by destroying nucleic acids and disrupting their DNA, leaving them unable to perform vital cellular functions. This is used in a variety of applications, such as purification of food, air, and/or water. A wavelength of 260 nm is particularly relevant in this respect as nucleic acids such as DNA have an absorption maximum at this wavelength. For example, the A260/A280 ratio is often used to indicate the purity of a DNA sample. “A260” refers to the absorption at a wavelength of 260 nm and “A280” refers to the absorption at a wavelength of 280 nm. A high A260/A280 ratio indicates a high purity of the DNA sample. The reason is that DNA strongly absorbs at 260 nm whereas protein contaminations mainly absorb at 280 nm. Thus, a high A260/A280 ratio means that the sample contains a high amount of DNA and a low amount of protein.

There are two main demands on glasses used in such lamps. First, the glasses should have a high UV transmittance, in particular in the UVC region. Second, the glasses should have a high solarization resistance so that their performance does not decrease too quickly upon use. Thus, a high solarization resistance increases the lifetime of the UV lamps. Consequently, there is a need for glasses combining both a high UV transmittance and a high solarization resistance.

Moreover, in view of the climate change, reducing consumption of fossil energy is more and more desired throughout different industries. In particular, the energy consumption during production of glasses is high. In order to improve the climate footprint of glass production, electrical energy can be used. Electrical energy may, for example, be produced from renewable energy sources such as wind energy or solar energy.

Using electrical energy, an electric current may be passed through the bath of molten glass between electrodes immersed in the molten glass. However, the choice of materials of the electrodes may be critical, in particular when glasses having high UV transmittance and high solarization resistance are concerned.

What is needed in the art is a way to overcome the disadvantages described above with respect to the state of the art by providing a way which allow a reduction of fossil energy consumption during production of glasses having high UV transmittance and high solarization resistance as well as provide such glass with particularly high quality.

### SUMMARY OF THE INVENTION

In some exemplary embodiments provided according to the present invention, a glass includes SiO₂ in an amount of at least 60.0 wt.-% and an amount of MoO₃ from 0.1 ppm to 30.0 ppm. Molybdenum is present in such oxidation states that a transmittance at a wavelength of 260 nm is at least 65.0% at a reference thickness of 1.0 mm.

In some exemplary embodiments provided according to the present invention, a lamp includes a glass including SiO₂ in an amount of at least 60.0 wt.-% and an amount of MoO₃ from 0.1 ppm to 30.0 ppm. Molybdenum is present in such oxidation states that a transmittance at a wavelength of 260 nm is at least 65.0% at a reference thickness of 1.0 mm.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates an emission spectrum of an HOK 4 lamp, on the x-axis the wavelength in nm is shown and on the y-axis the relative intensity in comparison to the maximum intensity is shown; and

FIG. 2 illustrates the transmittance spectra of exemplary glasses in a wavelength range of from 200 nm to 400 nm before and after irradiation with the HOK 4 lamp for 144 hours.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

For obtaining glass articles having high UV transmittance and high solarization resistance, so far technologies associated with high consumption of fossil energy had to be used.

However, exemplary embodiments provided according to the present invention provide a glass having high UV transmittance and high solarization resistance that can be obtained with reduced consumption of fossil energy without substantially compromising UV transmittance and solarization resistance. Some exemplary embodiments provided according to the invention are based on the finding that molybdenum electrodes can be used during production and that a certain amount of molybdenum emanating from the electrodes into the melt can be tolerated if reducing melting conditions are applied.

In some embodiments, the invention relates to a glass comprising SiO₂ in an amount of at least 60.0 wt.-%, wherein the amount of MoO₃ is from 0.1 ppm to 30.0 ppm, and wherein molybdenum is present in such oxidation states that the transmittance at a wavelength of 260 nm is at least 65% (at a reference thickness of 1.0 mm).

The present disclosure refers to molybdenum present in the glasses as “MoO₃”. However, that is not supposed to mean that molybdenum is present in the glass predominantly as molybdenum (VI). Rather, the term “MoO₃” simply follows the common way of referring to molybdenum in the glass and refers to the total amount of molybdenum irrespective of the oxidation state. The present invention includes reducing melting conditions. Thus, in some embodiments provided according to the invention the amount of molybdenum (VI) in the glasses is rather low. Molybdenum (VI) may not be the predominant molybdenum species in the glass. In the glasses provided according to the invention, molybdenum is present in such oxidation states that the transmittance at a wavelength of 260 nm is at least 65% (at a reference thickness of 1.0 mm).

In some embodiments, the invention relates to a glass comprising the following components in the indicated amounts (in wt.-%):

| Component | Amount (wt.-%) |
|---|---|
| SiO₂ | 60.0-80.0 |
| B₂O₃ | 0.5-30.0 |
| Al₂O₃ | 0.5-10.0 |
| Na₂O | 2.0-20.0 |
| K₂O | 0-20.0 |
| BaO | 0-15.0 |

In some embodiments, the partial pressure of oxygen (pO₂) in the glass melt at a temperature of 1500° C. is 0.5 bar or less when the glass melt is produced from the glass by inductively heating to a temperature of 1500° C. in a platinum crucible under argon atmosphere. The pO₂ at 1500° C. may, for example, be at most 0.4 bar, at most 0.3 bar, or at most 0.2 bar. In some embodiments, the pO₂ may, for example, be at least 0.01 bar, at least 0.02 bar, at least 0.05 bar, or at least 0.1 bar. The pO₂ may, for example, be from 0.01 bar to 0.5 bar, from 0.02 bar to 0.4 bar, from 0.05 bar to 0.3 bar, or from 0.1 bar to 0.2 bar. A comparably low pO₂ is advantageous as is associated with lower oxidation states of molybdenum. However, the pO₂ should not be too low in order to avoid occurrence of metallic molybdenum.

The pO₂ of the glass melt may, for example, be determined based on the voltage between a reference electrode and a measuring electrode both of which are positioned in the glass melt. The pO₂ can be calculated from the voltage between the electrodes by using the Nernst equation. A platinum plate can be used as measuring electrode. The reference electrode may comprise a platinum wire positioned inside a ZrO₂ ceramic tube that is closed at the tip, wherein the platinum wire is in electrically conducting contact with the wall of the ZrO₂ tube. The ZrO₂ ceramic may be yttrium-stabilized, calcium-stabilized, or magnesium-stabilized (see for example EP 1 101 740 A1, par. [0012], [0013]). For the measurement of the pO₂ of the glass melt, pure oxygen is flowing around the platinum wire so that there is a constant pO₂ of 1.0 bar at the platinum wire inside the reference electrode. The ZrO₂ is an oxygen conductor and forms a bridge between the platinum inside the reference electrode and the glass melt, and thus indirectly also with the platinum measuring electrode inside the glass melt. Oxygen ions migrate. The concentration cell “platinum (pO₂=constant=1.0 bar)/ZrO₂/glass melt/platinum (pO₂ of the glass melt)” generates a voltage. The voltage between the reference electrode and the measuring electrode is proportional to the pO₂ in the glass melt and can thus be converted to determine the pO₂ in the glass melt based on the Nernst equation.

The glass may comprise the following components in the indicated amounts (in wt.-%):

| Component | Amount (wt.-%) |
|---|---|
| SiO₂ | 60.0-75.0 |
| B₂O₃ | 0.5-15.0 |
| Al₂O₃ | 0.5-7.5 |
| Na₂O | 2.5-15.0 |
| K₂O | 2.0-16.0 |
| BaO | 2.5-12.5 |

The glass may comprise the following components in the indicated amounts (in wt.-%):

| Component | Amount (wt.-%) |
|---|---|
| SiO₂ | 60.0-75.0 |
| B₂O₃ | 0.5-5.0 |
| Al₂O₃ | 0.5-5.0 |
| Na₂O | 7.0-10.0 |
| K₂O | 8.0-12.0 |
| BaO | 4.5-10.0 |

In some embodiments, the glass provided according to the invention has a transmittance at a wavelength of 260 nm of at least 65.0%, for example at least 66.0%, at least 67.0%, at least 68.0%, at least 69.0%, at least 70.0%, at least 71.0%, at least 72.0%, at least 73.0%, at least 74.0%, at least 75.0%, at least 76.0%, at least 77.0%, at least 78.0%, at least 79.0%, or at least 80.0%. Transmittance values in the present disclosure refer to a reference thickness of 1.0 mm of the glass if not indicated otherwise. That does not mean that the glass necessarily has to have a thickness of 1.0 mm. Rather, the reference thickness simply indicates what the transmittance would be if the glass had a thickness of 1.0 mm. The transmittance at a reference thickness may be determined by measuring the transmittance of a sample having a thickness of 1.0 mm. Alternatively, the transmittance at a thickness of 1.0 mm may also be determined by measuring the transmittance at another sample thickness, for example at a sample thickness of 0.7 mm, and then extrapolating what the transmittance would be at a thickness of 1.0 mm. Generally speaking, the extrapolation of a transmittance T1 at a thickness d1 to a transmittance T2 at a thickness d2 can be done using the following formula: T2=((T1/P)\hat{ }(d2/d1))*P, wherein P is the wavelength-dependent reflection coefficient (P=P(λ)) given in the unit “%”. P can be determined via the Sellmeier n coefficient.

In some embodiments, the transmittance at a wavelength of 260 nm may be at most 99.0%, at most 97.5%, at most 95.0%, at most 94.0%, at most 93.0%, at most 92.0%, at most 91.0%, at most 90.0%, at most 89.0%, at most 88.0%, at most 87.0%, at most 86.0%, at most 85.0%, at most 84.0%, at most 83.0%, or at most 82.0% (at a reference thickness of 1.0 mm). The transmittance at a wavelength of 260 nm may, for example, be in a range of from 65.0% to 99.0%, from 66.0% to 97.5%, from 67.0% to 95.0%, from 68.0% to 94.0%, from 69.0% to 93.0%, from 70.0% to 92.0%, from 71.0% to 91.0%, from 72.0% to 90.0%, from 73.0% to 89.0%, from 74.0% to 88.0%, from 75.0% to 87.0%, from 76.0% to 86.0%, from 77.0% to 85.0%, from 78.0% to 84.0%, from 79.0% to 83.0%, or from 80.0% to 82.0%.

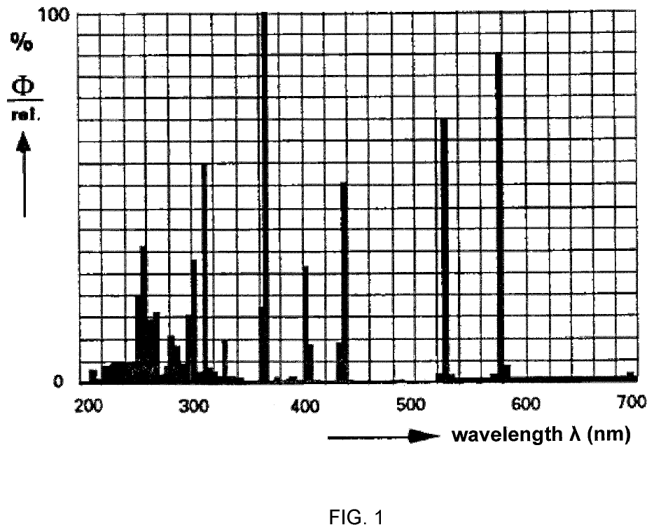

The glasses provided according to the invention are characterized by a particularly high solarization resistance. The solarization resistance can be determined by irradiating the glass with a HOK 4 lamp for 144 hours and comparing the transmittance (at a reference thickness of 1.0 mm) at a wavelength of 260 nm before and after irradiation. The term “HOK 4 lamp” refers to the high pressure mercury-vapor lamp HOK 4/120 of Phillips. The emission spectrum of the HOK 4 lamp is shown in FIG. 1. The main emission of the lamp is at a wavelength of 365 nm. The power density at 200-280 nm in a distance of 1 m is 850 μW/cm². For the irradiation for 144 hours according to the present invention, the distance between the HOK 4 lamp and the sample is chosen to be 7 cm.

The lower the difference between the transmittance before and after irradiation is, the higher is the solarization resistance. For example, there may be two glasses, each having a transmittance (at a reference thickness of 1.0 mm) of 80% at a wavelength of 260 nm (before irradiation). After irradiation with the HOK 4 lamp for 144 hours, the transmittance may be 75% for the first glass and 70% for the second glass. Thus, the difference between transmittance (at a reference thickness of 1.0 mm) at a wavelength of 260 nm prior to irradiation with the HOK 4 lamp and transmittance (at a reference thickness of 1.0 mm) at a wavelength of 260 nm after irradiation with the HOK 4 lamp for 144 hours is 5% for the first glass and 10% for the second glass. Hence, the solarization resistance of the first glass is higher as compared to the second glass because the difference between the transmittance before and after irradiation is lower for the first glass as compared to the second glass. A high solarization resistance is associated with low solarization and vice versa. A high solarization correlates with a high induced extinction Ext_{ind}.

The induced extinction Ext_{ind} can be determined based on the transmittance before and after irradiation with the HOK 4 lamp for 144 hours and the thickness of the glass sample using the following formula:

$${Ext}_{ind} = - \frac{ln \frac{{T}_{after}}{{T}_{before}}}{d}$$

Ext_{ind} is the induced extinction, T_{after} is the transmittance after irradiation with the HOK 4 lamp for 144 hours, T_{before} is the transmittance before irradiation with the HOK 4 lamp for 144 hours, d is the sample thickness, and ln is the natural logarithm. If not indicated otherwise, the sample thickness d is given in cm so that the induced extinction is given in 1/cm. If not indicated otherwise, the transmittance before and after irradiation with the HOK 4 lamp are given for a wavelength of 260 nm. Thus, the induced extinction as described in the present disclosure refers to the induced extinction at a wavelength of 260 nm if not indicated otherwise.

In some embodiments, the induced extinction at a wavelength of 260 nm is at most 3.5/cm, at most 3.2/cm, at most 3.0/cm, at most 2.9/cm, at most 2.8/cm, at most 2.7/cm, at most 2.6/cm, at most 2.5/cm, at most 2.4/cm, at most 2.3/cm, at most 2.2/cm, at most 2.1/cm, at most 2.0/cm, at most 1.9/cm, at most 1.8/cm, at most 1.7/cm, at most 1.6/cm, at most 1.5/cm, at most 1.4/cm, or at most 1.3/cm. The induced extinction at a wavelength of 260 nm may, for example, be at least 0.01/cm, at least 0.02/cm, at least 0.05/cm, at least 0.1/cm, at least 0.2/cm, at least 0.3/cm, at least 0.4/cm, at least 0.5/cm, at least 0.55/cm, at least 0.6/cm, at least 0.65/cm, at least 0.7/cm, at least 0.75/cm, at least 0.8/cm, at least 0.85/cm, at least 0.9/cm, at least 0.95/cm, at least 1.0/cm, at least 1.05/cm, or at least 1.1/cm. The induced extinction at a wavelength of 260 nm may, for example, be from 0.01/cm to 3.5/cm, from 0.02/cm to 3.2/cm, from 0.05/cm to 3.0/cm, from 0.1/cm to 2.0/cm, from 0.2/cm to 2.8/cm, from 0.3/cm to 2.7/cm, from 0.4/cm to 2.6/cm, from 0.5/cm to 2.5/cm, from 0.55/cm to 2.4/cm, from 0.6/cm to 2.3/cm, from 0.65/cm to 2.2/cm, from 0.7/cm to 2.1/cm, from 0.75/cm to 2.0/cm, from 0.8/cm to 1.9/cm, from 0.85/cm to 1.8/cm, from 0.9/cm to 1.7/cm, from 0.95/cm to 1.6/cm, from 1.0/cm to 1.5/cm, from 1.05/cm to 1.4/cm, or from 1.1/cm to 1.3/cm.

In some embodiments, the glass provided according to the invention is characterized by a high UV transmittance and a low induced extinction. This combination is associated with a high transmittance (at a reference thickness of 1.0 mm) at a wavelength of 260 nm after irradiation with the HOK 4 lamp for 144 hours. The transmittance (at a reference thickness of 1.0 mm) at a wavelength of 260 nm after irradiation with the HOK 4 lamp for 144 hours may, for example, be at least 54.0%, at least 55.0%, at least 56.0%, at least 57.0%, at least 58.0%, at least 59.0%, at least 60.0%, at least 61.0%, at least 62.0%, at least 63.0%, at least 64.0%, at least 65.0%, at least 66.0%, at least 67.0%, at least 68.0%, at least 69.0%, at least 70.0%, at least 71.0%, at least 72.0%, at least 73.0%, or at least 74.0%.

The transmittance (at a reference thickness of 1.0 mm) at a wavelength of 260 nm after irradiation with the HOK 4 lamp for 144 hours may, for example, be at most 95.0%, at most 94.0%, at most 93.0%, at most 92.0%, at most 91.0%, at most 90.0%, at most 89.0%, at most 88.0%, at most 87.0%, at most 86.0%, at most 85.0%, at most 84.0%, at most 83.0%, at most 82.0%, at most 81.0%, at most 80.0%, at most 79.0%, at most 78.0%, at most 77.0%, at most 76.0%, or at most 75.0%. The transmittance at a wavelength of 260 nm may, for example, be in a range of from 54.0% to 95.0%, from 55.0% to 94.0%, from 56.0% to 93.0%, from 57.0% to 92.0%, from 58.0% to 91.0%, from 59.0% to 90.0%, from 60.0% to 89.0%, from 61.0% to 88.0%, from 62.0% to 87.0%, from 63.0% to 86.0%, from 64.0% to 85.0%, from 65.0% to 84.0%, from 66.0% to 83.0%, from 67.0% to 82.0% from 68.0% to 81.0%, from 69.0% to 80.0%, from 70.0% to 79.0%, from 71.0% to 78.0%, from 72.0% to 77.0%, from 73.0% to 76.0%, or from 74.0% to 75.0%.

Molybdenum (Mo) electrodes are subject to corrosion and removal of electrode material during use. Molybdenum emanating from the electrodes can then also be found in the molten glass material. This leads to contaminations of the final glass articles produced from the molten glass. Such contaminations are in turn associated with compromised properties of the resulting glasses, in particular with reduced UV transmittance and/or reduced solarization resistance. The present invention includes the finding that molybdenum electrodes can be used during production and that a certain amount of molybdenum emanating from the electrodes into the melt can be tolerated if reducing melting conditions are applied.

The glass provided according to the invention comprises MoO₃ in an amount of from 0.1 ppm to 30.0 ppm. The amount of MoO₃ may, for example, be at least 0.2 ppm, at least 0.4 ppm, at least 0.5 ppm, at least 1.0 ppm, at least 2.0 ppm, at least 3.0 ppm, or at least 5.0 ppm. The amount of MoO₃ may, for example, be at most 25.0 ppm, at most 22.5 ppm, at most 20.0 ppm, at most 17.5 ppm, at most 15.0 ppm, at most 12.5 ppm, or at most 10.0 ppm. The amount of MoO₃ may, for example, be from 0.2 ppm to 25.0 ppm, from 0.4 ppm to 22.5 ppm, from 0.5 ppm to 20.0 ppm, from 1.0 ppm to 17.5 ppm, from 2.0 ppm to 15.0 ppm, from 3.0 ppm to 12.5 ppm, or from 5.0 ppm to 10.0 ppm.

The glass provided according to the invention may comprise SiO₂ in an amount of from 60.0 to 80.0 wt.-%, for example from 60.0 to 75.0 wt.-% or from 62.5 to 75.0 wt.-%. The amount of SiO₂ is at least 60.0%, for example at least 62.5%. The amount of SiO₂ may, for example, be at most 80.0 wt.-% or at most 75.0 wt.-%.

The glass provided according to the invention may comprise B₂O₃ in an amount of from 0.5 to 30.0 wt.-%, for example from 0.5 to 25.0 wt.-%, from 0.5 to 20.0 wt.-%, from 0.5 to 15.0 wt.-%, from 0.5 to 10.0 wt.-%, from 0.5 to 5.0 wt.-% or from 1.0 to 4.0 wt.-%. The amount of B₂O₃ may, for example, be at least 0.5 wt.-% or at least 1.0 wt.-%. The amount of B₂O₃ may, for example, be at most 30.0 wt.-%, at most 25.0 wt.-%, at most 20.0 wt.-%, at most 15.0 wt.-%, at most 10.0 wt.-%, at most 5.0 wt.-% or at most 4.0 wt.-%.

The glass provided according to the invention may comprise Al₂O₃ in an amount of from 0.5 to 10.0 wt.-%, for example from 0.5 to 7.5 wt.-%, from 0.5 to 5.0 wt.-% or from 1.0 wt.-% to 4.5 wt.-%. The amount of Al₂O₃ may, for example, be at least 0.5 wt.-% or at least 1.0 wt.-%. The amount of Al₂O₃ may, for example, be at most 10.0 wt.-%, at most 7.5 wt.-%, at most 5.0 wt.-% or at most 4.5 wt.-%.

The glass provided according to the invention may comprise Na₂O in an amount of from 2.0 to 20.0 wt.-%, from 2.25 to 19.0 wt.-%, or from 2.5 to 18.0 wt.-%, from 5.0 wt.-% to 17.5 wt.-%, from 6.0 wt.-% to 15.0 wt.-% or from 7.0 to 10.0 wt.-%. The amount of Na₂O may, for example, be at least 2.0 wt.-%, at least 2.25 wt.-%, at least 2.5 wt.-%, at least 5.0 wt.-%, at least 6.0 wt.-% or at least 7.0 wt.-%. The amount of Na₂O may, for example, be at most 20.0 wt.-%, at most 19.0 wt.-%, at most 18.0 wt.-%, at most 17.5 wt.-%, at most 15.0 wt.-% or at most 10.0 wt.-%.

The glass provided according to the invention may comprise K₂O in an amount of from 0 to 20.0 wt.-% for example from 1.0 to 18.0 wt.-%, from 2.0 to 16.0 wt.-%, from 5.0 to 14.0 wt.-% or from 8.0 to 12.0 wt.-%. The amount of K₂O may, for example, be at least 1.0 wt.-%, at least 2.0 wt.-%, at least 5.0 wt.-% or at least 8.0 wt.-%. The amount of K₂O may, for example, be at most 20.0 wt.-%, at most 18.0 wt.-%, at most 16.0 wt.-%, at most 14.0 wt.-% or at most 12.0 wt.-%. In some embodiments, the amount of K₂O is at most 1.0 wt.-%, at most 0.5 wt.-%, at most 0.2 wt.-% or at most 0.1 wt.-%.

The glass provided according to the invention may comprise Li₂O. The amount of Li₂O may, for example, be in an amount of from 0 to 5.0 wt.-%, for example from 0.1 to 2.0 wt.-% or from 0.5 to 1.5 wt.-%. The amount of Li₂O may, for example, be at least 0.1 wt.-%, at least 0.2 wt.-% or at least 0.5 wt.-%. The amount of Li₂O may, for example, be at most 5.0 wt.-%, at most 2.0 wt.-% or at most 1.5 wt.-%. In some embodiments, the amount of Li₂O is at most 1.0 wt.-%, at most 0.5 wt.-%, at most 0.2 wt.-% or at most 0.1 wt.-%.

In some embodiments, the sum of the amounts of Li₂O, Na₂O and K₂O is in a range of from 4.0 wt.-% to 30.0 wt.-% or from 5.0 to 30.0 wt.-%, for example from 7.5 wt.-% to 27.5 wt.-%, from 10.0 wt.-% to 25.0 wt.-%, from 12.5 wt.-% to 22.5 wt.-% or from 15.0 wt.-% to 20.0 wt.-%. The sum of the amounts of Li₂O, Na₂O and K₂O may, for example, be at least 4.0 wt.-%, at least 5.0 wt.-%, at least 7.5 wt.-%, at least 10.0 wt.-%, at least 12.5 wt.-% or at least 15.0 wt.-%. The sum of the amounts of Li₂O, Na₂O and K₂O may, for example, be at most 30.0 wt.-%, at most 27.5 wt.-%, at most 25.0 wt.-%, at most 22.5 wt.-% or at most 20.0 wt.-%.

In some embodiments, the sum of the amounts of Na₂O and K₂O is in a range of from 3.5 wt.-% to 30.0 wt.-% or from 5.0 to 30.0 wt.-%, for example from 7.5 wt.-% to 27.5 wt.-%, from 10.0 wt.-% to 25.0 wt.-%, from 12.5 wt.-% to 22.5 wt.-% or from 15.0 wt.-% to 20.0 wt.-%. The sum of the amounts of Na₂O and K₂O may, for example, be at least 3.5 wt.-%, at least 5.0 wt.-%, at least 7.5 wt.-%, at least 10.0 wt.-%, at least 12.5 wt.-% or at least 15.0 wt.-%. The sum of the amounts of Na₂O and K₂O may, for example, be at most 30.0 wt.-%, at most 27.5 wt.-%, at most 25.0 wt.-%, at most 22.5 wt.-% or at most 20.0 wt.-%.

The glasses provided according to the invention may comprise BaO in an amount of from 0 to 15.0 wt.-%, for example from 2.5 to 12.5 wt.-%, from 4.5 to 10.0 wt.-% or from 5.0 to 8.0 wt.-%. The amount of BaO may, for example, be at least 2.5 wt.-%, at least 4.5 wt.-% or at least 5.0 wt.-%. The amount of BaO may, for example, be at most 15.0 wt.-%, at most 12.5 wt.-%, at most 10.0 wt.-% or at most 8.0 wt.-%. In some embodiments, the amount of BaO is at most 1.0 wt.-%, at most 0.5 wt.-%, at most 0.2 wt.-% or at most 0.1 wt.-%.

The glass may consist of SiO₂, B₂O₃, Al₂O₃, Na₂O, K₂O and BaO to an extent of at least 95.0 wt.-%, at least 97.0 wt.-%, or at least 99.0 wt.-%.

In some embodiments, the amount of Fe₂O₃ is less than 100 ppm, less than 50 ppm, less than 20 ppm, less than 15 ppm, or less than 10 ppm.

In some embodiments, the amount of TiO₂ is less than 100 ppm, less than 50 ppm, less than 20 ppm, less than 15 ppm, or less than 10 ppm.

In some embodiments, the amount of NiO is less than 100 ppm, less than 50 ppm, less than 20 ppm, less than 15 ppm, or less than 10 ppm.

In some embodiments, the amount of Cr₂O₃ is less than 100 ppm, less than 50 ppm, less than 20 ppm, less than 15 ppm, less than 10 ppm, or less than 5 ppm.

In some embodiments, the amount of at least one, at least two, or at least three of Fe₂O₃, TiO₂, NiO and Cr₂O₃ is less than 100 ppm, less than 50 ppm, less than 20 ppm, less than 15 ppm, or less than 10 ppm.

In some embodiments, the amount of Fe₂O₃, TiO₂, NiO and Cr₂O₃ is less than 100 ppm, less than 50 ppm, less than 20 ppm, less than 15 ppm, or less than 10 ppm.

The term “ppm” as used herein refers to ppm on a weight basis if not indicated otherwise. For example, 1000 ppm correspond to 0.1 wt.-%.

In some embodiments, the amount of ZrO₂ is less than 1.0 wt.-%, less than 0.5 wt.-%, less than 0.2 wt.-% or less than 0.1 wt.-%.

The glass provided according to the invention may comprise Cl in an amount of more than 100 ppm, more than 200 ppm or more than 500 ppm, for example at least 0.1 wt.-%. In some embodiments, the amount of Cl is at most 1.5 wt.-%, at most 1.2 wt.-%, at most 1.0 wt.-% or at most 0.8 wt.-%. The amount of Cl may, for example, be from 100 ppm to 1.5 wt.-%, from 200 ppm to 1.2 wt.-%, from 500 ppm to 1.0 wt.-% or from 0.1 wt.-% to 0.8 wt.-%.

In some embodiments provided according to the present invention, the glass has a composition that makes it particularly resistant against the influence of MoO₃ on UV transmittance and/or solarization. The glass may have a good UV transmittance and a good solarization resistance even though certain amounts of MoO₃ are present in the glass. A certain amount of MoO₃ can be accepted in the glasses provided according to the invention, in particular due to the reducing melting conditions applied.

Increasing amounts of MoO₃ are associated with decreasing transmittance at a wavelength of 260 nm. However, in some embodiments provided according to the present invention the glass composition is such that molybdenum induced extinction is low. Therefore, higher amounts of MoO₃ then expected may be acceptable in the glasses provided according to the present invention.

The molybdenum induced extinction MoExt_{ind} can be determined based on the transmittance before and after irradiation with the HOK 4 lamp for 144 hours, the thickness of the glass sample, and the amount of MoO₃ using the following formula:

$${MoExt}_{ind} = - \frac{ln \frac{{T}_{after}}{{T}_{before}}}{d \cdot Mo}$$

MoExt_{ind} is the molybdenum induced extinction, T_{after} is the transmittance after irradiation with the HOK 4 lamp for 144 hours, T_{before} is the transmittance before irradiation with the HOK 4 lamp for 144 hours, d is the sample thickness, ln is the natural logarithm, and Mo is the amount of MoO₃ in ppm. If not indicated otherwise, the sample thickness d is given in cm so that the molybdenum induced extinction is given in 1/(cm*ppm). If not indicated otherwise, the transmittance before and after irradiation with the HOK 4 lamp is given for a wavelength of 260 nm. Thus, the molybdenum induced extinction as described in the present disclosure refers to the molybdenum induced extinction at a wavelength of 260 nm if not indicated otherwise.

In some embodiments, the molybdenum induced extinction at a wavelength of 260 nm is at most 0.20/(cm*ppm), at most 0.15/(cm*ppm), at most 0.14/(cm*ppm), at most 0.13/(cm*ppm), at most 0.12/(cm*ppm), at most 0.11/(cm*ppm), or at most 0.10/(cm*ppm) at a reference amount of MoO₃ of 20 ppm. The molybdenum induced extinction at a wavelength of 260 nm may, for example, be at least 0.01/(cm*ppm), at least 0.02/(cm*ppm), at least 0.03/(cm*ppm), at least 0.04/(cm*ppm), at least 0.05/(cm*ppm), at least 0.06/(cm*ppm), or at least 0.07/(cm*ppm) at a reference amount of MoO₃ of 20 ppm. The molybdenum induced extinction at a wavelength of 260 nm may, for example, be from 0.01/(cm*ppm) to 0.20/(cm*ppm), from 0.02/(cm*ppm) to 0.15/(cm*ppm), from 0.03/(cm*ppm) to 0.14/(cm*ppm), from 0.04/(cm*ppm) to 0.13/(cm*ppm), from 0.05/(cm*ppm) to 0.12/(cm*ppm), from 0.06/(cm*ppm) to 0.11/(cm*ppm), or from 0.07/(cm*ppm) to 0.10/(cm*ppm) at a reference amount of MoO₃ of 20 ppm.

The invention also relates to a method of producing a glass provided according to the invention, the method comprising the following steps:
a) Melting glass raw materials, the melting step comprising the use of molybdenum electrodes,
b) Optionally refining the melt,
c) Cooling the melt.

The method is characterized by reducing melting conditions such that molybdenum is present in such oxidation states that the transmittance at a wavelength of 260 nm is at least 65% (at a reference thickness of 1.0 mm). Such conditions result in the compromising influence of molybdenum on UV transmittance and solarization resistance to be drastically reduced so that a certain amount of molybdenum emanating from the electrodes into the melt can be tolerated without substantially compromising UV transmittance and solarization resistance. The reducing character of the melt is also influenced by the temperature. The method may comprise temperatures of 1440° C. or more, for example 1500° C. or more. The viscosity at a given temperature is dependent on the glass composition. The method provided according to the invention may involve viscosities of 2.5 dPas or less.

However, the conditions should not be too reducing in order to avoid metallic molybdenum.

Providing reducing melting conditions may comprise adding one or more reducing agent, for example one or more carbohydrate, in particular one or more sugar, for example sucrose, to the glass raw materials, in particular in an amount of from 0.1 to 1.0 wt.-%, for example from 0.2 to 0.6 wt.-%. Glass raw materials comprising nitrates may be avoided. In some embodiments, the amount of nitrates is at most 200 ppm, at most 100 ppm, at most 50 ppm, at most 20 ppm, at most 10 ppm, at most 5 ppm, at most 2 ppm, or at most 1 ppm.

The optional refining step may comprise using Cl as refining agent. Cl is an exemplary refining agent as it has a reducing character as well. Oxidizing refining agents may be avoided. In some embodiments, the amount of oxidizing refining agents is at most 200 ppm, at most 100 ppm, at most 50 ppm, at most 20 ppm, at most 10 ppm, at most 5 ppm, at most 2 ppm, or at most 1 ppm.

The invention also relates to the use of glass provided according to the invention for or as UVC detectors, envelope in germicidal lamps, UV-LED (in particular UVC-LED), UV-transmitting lamps, protective tubing for UV-lamps, UV-transmitting material for UV oxidation reactors, UV flame detectors, UV photoelectric cells, sun reactors, spectral analysis device, photomultiplier, and for windows (in particular EPROM windows), cover plate for solar cells (e.g. in space), UV-transmitting cuvette (e.g. for photoluminescence measurements with UV excitation), UV-CCL (cold cathode lamps) and/or xenon flash lamps.

In some embodiments, the invention relates to the use of the glass provided according to the invention in diagnostics, in particular for or as microfluidic component, e.g. for photoluminescence-based diagnostics. The glass may be used for or as the bottom plate or the cover plate of a microfluidic component. The high UV-transmittance is advantageous for the signal-to-noise ratio of the diagnostic method.

In some embodiments, the invention relates to the use of the glass provided according to the invention for lamps emitting UV light, in particular UV lamps with or without protective tubing.

In some embodiments, the invention relates to the use of a glass provided according to the invention in UVC detectors or as envelope in germicidal lamps.

The invention also relates to a UVC detector or a germicidal lamp comprising the glass provided according to the invention.

### EXAMPLES

### 1. Example A

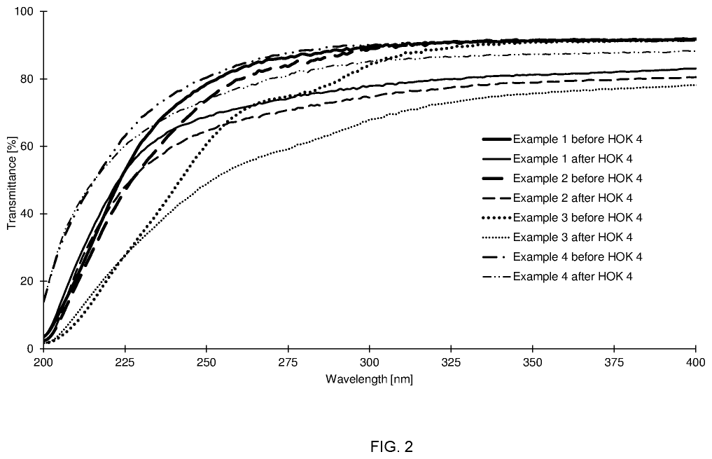

Alkali-containing silicate glasses in accordance with the glass composition as described in the present disclosure have been tested with respect to UV transmittance and solarization resistance. The composition of the glasses merely differed with respect to the amount of MoO₃. The amount of MoO₃ was 7.4 ppm in Example 1, 23 ppm in Example 2, 59 ppm in Example 3, and less than 3 ppm in Example 4. All examples were obtained under reducing melting conditions by adding sucrose to the glass raw materials in an amount of 0.4 wt.-%. Samples having a thickness of about 0.7 mm have been tested for transmittance in the wavelength range of from 200 nm to 400 nm before and after irradiation with the HOK 4 lamp. The results are shown in FIG. 2.

In particular, regarding the particularly relevant wavelength of 260 nm, the results obtained are summarized in the following table.

| | Transmittance at a | Transmittance at a | |
|---|---|---|---|
| | thickness of 0.7 mm | thickness of 0.7 mm | Induced |
| | before HOK 4 | after HOK 4 | Extinction |
| | irradiation [%] | irradiation [%] | [1/cm] |
| Example 1 | 82.9 | 71.3 | 2.15 |
| Example 2 | 79.8 | 67.6 | 2.37 |
| Example 3 | 69.9 | 54.4 | 3.58 |
| Example 4 | 84.2 | 77.4 | 1.20 |

The results show that an increasing amount of MoO₃ has two different effects. On the one hand, the transmittance is reduced. On the other hand, the induced extinction is increased.

### 2. Example B

Alkali-containing silicate glasses in accordance with the glass composition as described in the present disclosure comprising MoO₃ in an amount of about 20 to 25 ppm have been tested with respect to UV transmittance and solarization resistance. The glasses of Examples 4 and 5 whose compositions were basically identical (23 ppm MoO₃ in Example 4 and 21 ppm MoO₃ in Example 5) mainly differed with respect to the melting conditions applied. Example 4 was obtained under reducing melting conditions by adding sucrose to the glass raw materials. In contrast, Example 5 was obtained under oxidizing melting conditions (1 wt.-% nitrate). Samples having a thickness of about 1.0 mm have been tested for transmittance at a wavelength of 260 nm before and after irradiation with the HOK 4 lamp. The results obtained are summarized in the following table.

| | Transmittance at a | Transmittance at a |
|---|---|---|
| | thickness of 1.0 mm before | thickness of 1.0 mm after |
| | HOK 4 irradiation [%] | HOK 4 irradiation [%] |
| Example 4 | 75.8 | 59.9 |
| Example 5 | 64.9 | 53.0 |

The transmittance at a wavelength of 260 nm was higher in Example 4 obtained under reducing melting conditions as compared to Example 5 obtained under oxidizing melting conditions both before and after irradiation with the HOK 4 lamp.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A glass, comprising SiO₂ in an amount of at least 60.0 wt.-% and an amount of MoO₃ from 0.1 ppm to 30.0 ppm, wherein molybdenum is present in such oxidation states that a transmittance at a wavelength of 260 nm is at least 65.0% at a reference thickness of 1.0 mm.

2. The glass of claim 1, comprising the following components in the indicated amounts (in wt.-%):

| Component | Amount (wt.-%) |
|---|---|
| SiO₂ | 60.0-80.0 |
| B₂O₃ | 0.5-30.0 |
| Al₂O₃ | 0.5-10.0 |
| Na₂O | 2.0-20.0 |
| K₂O | 0-20.0 |
| BaO | 0-15.0. |

3. The glass of claim 1, comprising the following components in the indicated amounts (in wt.-%):

| Component | Amount (wt.-%) |
|---|---|
| SiO₂ | 60.0-75.0 |
| B₂O₃ | 0.5-5.0 |
| Al₂O₃ | 0.5-5.0 |
| Na₂O | 7.0-10.0 |
| K₂O | 8.0-12.0 |
| BaO | 4.5-10.0. |

4. The glass of claim 1, wherein the glass comprises less than 10 ppm Fe₂O₃.

5. The glass of claim 1, wherein the glass comprises less than 10 ppm TiO₂.

6. The glass of claim 1, wherein the glass comprises less than 10 ppm NiO.

7. The glass of claim 1, wherein the glass comprises less than 10 ppm Cr₂O₃.

8. The glass of claim 1, wherein the glass comprises Cl in an amount of more than 100 ppm.

9. The glass of claim 1, wherein a proportion of ZrO₂ in the glass is less than 0.5 wt.-%.

10. The glass of claim 1, wherein a transmittance at a reference thickness of 1.0 mm and a wavelength of 260 nm after irradiation with a HOK 4 lamp for 144 hours is at least 54.0%.

11. The glass of claim 1, wherein an induced extinction (Ext_{ind}) at a wavelength of 260 nm is at most 3.5/cm, wherein the induced extinction Ext_{ind} is determined based on a transmittance before and after irradiation with a HOK 4 lamp for 144 hours and a sample thickness of the glass using the following formula:
$${Ext}_{ind} = - \frac{ln \frac{{T}_{after}}{{T}_{before}}}{d} ,$$
wherein Ext_{ind} is the induced extinction, T_{after} is the transmittance after irradiation with the HOK 4 lamp for 144 hours, T_{before} is the transmittance before irradiation with the HOK 4 lamp for 144 hours, d is the sample thickness, and ln is the natural logarithm.

12. The glass of claim 1, wherein a molybdenum induced extinction (MoExt_{ind}) at a wavelength of 260 nm is at most 0.20/(cm*ppm) at a reference amount of MoO₃ of 20 ppm, wherein the molybdenum induced extinction MoExt_{ind} is determined based on a transmittance before and after irradiation with the HOK 4 lamp for 144 hours, a sample thickness of the glass, and the amount of MoO3 using the following formula:
$${MoExt}_{ind} = - \frac{ln \frac{{T}_{after}}{{T}_{before}}}{d \cdot Mo} ,$$
wherein MoExt_{ind} is the molybdenum induced extinction, T_{after} is the transmittance after irradiation with the HOK 4 lamp for 144 hours, T_{before} is the transmittance before irradiation with the HOK 4 lamp for 144 hours, d is the sample thickness, ln is the natural logarithm, and Mo is the amount of MoO₃ in ppm.

13. The glass of claim 1, wherein a partial pressure of oxygen (pO₂) in a glass melt at a temperature of 1500° C. is 0.5 bar or less, when the glass melt is produced from the glass by inductively heating to a temperature of 1500° C. in a platinum crucible under argon atmosphere.

14. A lamp, comprising:
a glass comprising SiO₂ in an amount of at least 60.0 wt.-% and an amount of MoO₃ from 0.1 ppm to 30.0 ppm, wherein molybdenum is present in such oxidation states that a transmittance at a wavelength of 260 nm is at least 65.0% at a reference thickness of 1.0 mm.

15. The lamp of claim 14, wherein the glass comprises the following components in the indicated amounts (in wt.-%):

| Component | Amount (wt.-%) |
|---|---|
| SiO₂ | 60.0-80.0 |
| B₂O₃ | 0.5-30.0 |
| Al₂O₃ | 0.5-10.0 |
| Na₂O | 2.0-20.0 |
| K₂O | 0-20.0 |
| BaO | 0-15.0. |

16. The lamp of claim 14, wherein the glass comprises the following components in the indicated amounts (in wt.-%):

| Component | Amount (wt.-%) |
|---|---|
| SiO₂ | 60.0-75.0 |
| B₂O₃ | 0.5-5.0 |
| Al₂O₃ | 0.5-5.0 |
| Na₂O | 7.0-10.0 |
| K₂O | 8.0-12.0 |
| BaO | 4.5-10.0. |

17. The lamp of claim 14, wherein the glass comprises less than 10 ppm Fe₂O₃.

18. The lamp of claim 14, wherein the glass comprises less than 10 ppm TiO₂.

19. The lamp of claim 14, wherein the glass comprises less than 10 ppm NiO.

20. The lamp of claim 14, wherein the glass comprises less than 10 ppm Cr₂O₃.
